(19) United States
(12) Patent Application Publication
(10) Pub. No.: US 2024/0182488 A1
(43) Pub. Date: Jun. 6, 2024

(54) Process for Preparing Isohexide Diamines From Isohexide Diols

(71) Applicants: Frank Richter, Leverkusen (DE); Raul Pires, Köln (DE); Serguei Kostromine, Swisttal-Buschhofen (DE)

(72) Inventors: Frank Richter, Leverkusen (DE); Raul Pires, Köln (DE); Serguei Kostromine, Swisttal-Buschhofen (DE)

(73) Assignee: Covestro Deutschland AG, Leverkusen (DE)

(21) Appl. No.: 18/285,868

(22) PCT Filed: Apr. 5, 2022
(86) PCT No.: PCT/EP2022/058977

§ 371 Date: Oct. 6, 2023

(30) Foreign Application Priority Data
Apr. 7, 2021 (EP) 21167174.8

(51) Int. Cl. C07D 493/04; C08G 18/32; C08G 18/72

(52) U.S. Cl. CPC C07D 493/04; C08G 18/3246; C08G 18/72

(57) ABSTRACT

The invention relates to a process for preparing isohexide diamines from isohexide diols, including the following steps: a) reacting at least one isohexide diol to give at least one suitably substituted derivative, b) aminating the at least one suitably substituted derivative from step a) to give a reaction product mixture, c) fractionating the reaction product mixture from step b) to give a substantially bis-aminated isohexide-containing product, d) deprotecting the substantially bis-aminated isohexide-containing product purified in step c), to give an isohexide diamine, and e) purifying the isohexide diamine from step d) to give a purified isohexide diamine, with step c) being carried out before step d).

### CROSS-REFERENCE TO RELATED APPLICATIONS

This application is the United States national phase of International Application No. PCT/EP2022/058977 filed Apr. 5, 2022, and claims priority to European Patent Application No. 21167174.8 filed Apr. 7, 2021, the disclosures of which are hereby incorporated by reference in their entireties.

### BACKGROUND

### Technical Field

The present invention relates to a process for preparing isohexide diamines from isohexide diols, to compositions comprising these isohexide diamines, and to the use of the compositions for polymer raw materials and/or polymers and to the polymer raw materials and/or polymers comprising such reaction products. Of particular importance is (3R,3aR,6S,6aR)-3,6-diamino-2,3,3a,5,6,6a-hexahydrofuro[3,2-b]furan (IUPAC name generated using the program BioVia/Draw, MDL.Draw.Editor 16.1.0.693; all further IUPAC names in the present text were created using the same program), which is also called “isosorbide diamine” or “ISODA” in the text below and which can be prepared from the dihydroxy compound of the same configuration, (3R,3aR,6S,6aR)-3,6-dihydroxy-2,3,3a,5,6,6a-hexahydrofuro[3,2-b]furan, trivial name: isosorbitol.

### Description of Related Art

Isosorbitol is industrially available in sufficient quantity, in good quality and with very high stereo purity for example from Roquette.

Angew. Chem. 2011, 123, 7741-7745; Angew. Chem. Int. Ed. 2011, 50, 7599-7603 (DOI: 10.1002/anie.201103199) describes a catalytic process inter alia for converting isohexide diols into the corresponding diamines. However, the selected reagents and reaction conditions make the process appear unsuitable for transfer to the multi-gram scale, let alone the multi-kg scale. Also, the reaction of the isomerically pure isosorbitol results in a mixture of the three possible isomeric diamines having the isosorbitol, isomannitol and isoiditol configuration.

A further process for preparing diamines from isohexide diols is described by S. Thiyagarajan, L. Gootjes, W. Vogelzang, J. van Haveren, M. Lutz, D. S. van Es, in ChemSusChem 2011, 4, 1823-1829 (DOI 10.1002/cssc.201100398).

This document describes a three-stage process on a laboratory scale for preparing isohexide-based (di)amines by means of consecutive tosylation, benzylation and subsequent catalytic debenzylation to form the (di)amine with precise control of the stereoisomer distribution in the final (di)amines. There is no disclosure of uses or applications of the diamines.

Forming the isomer having the isomannitol configuration should be avoided as far as possible since this yields, by way of an intramolecular subsequent reaction, in particular the tricyclic 2,5-(amino)-2,5-dideoxy-1,4:3,6-dianhydromannitol, which can only be separated from the target compound with considerable technical complexity. As a secondary amine, the latter functions as a chain terminator when using ISODA for polymer applications. Alternatively, when converted into the corresponding diisocyanate by means of phosgenation, the tricyclic 2,5-(chlorocarbamoyl)-2,5-dideoxy-1,4:3,6-dianhydromannitol would result as conversion product and would be an extremely troublesome impurity on account of the high AC/HC values of the resulting diisocyanate that are to be expected.

Distillative removal of the tricyclic 2,5-(amino)-2,5-dideoxy-1,4:3,6-dianhydromannitol to residual contents significantly below 1000 ppm by weight, even better 100 ppm by weight, in order to convert diamines into the corresponding diisocyanates by means of phosgenation, is not possible with a technically acceptable level of complexity.

Furthermore, the presence of N-benzylpyrrole, which is not mentioned in ChemSusChem 2011, 4, 1823-1829 and is just as difficult to remove from the target molecule by distillation as the abovementioned tricyclic monoamine, is also disadvantageous in the preparation and/or further processing of the resulting diisocyanate and in the case of other reactions of the diamine.

### SUMMARY

The object of the present invention was therefore that of providing a process, which can also be realized on an industrial scale and can be performed without complex separation techniques such as column chromatography, for preparing high-quality isohexide diamines, preferably ISODA, which should in particular be free of chain-terminating monoamines and/or troublesome further secondary components such as tertiary amines, particularly N-benzylpyrrole.

This object was achieved by a process for preparing isohexide diamines from isohexide diols, comprising the following steps:
a) reacting at least one isohexide diol to obtain at least one suitably substituted derivative,
b) aminating the at least one suitably substituted derivative from step a) to obtain a reaction product mixture,
c) separating the reaction product mixture from step b) to obtain a substantially bis-aminated isohexide-containing product,
d) deprotecting the substantially bis-aminated isohexide-containing product purified in step c) to obtain an isohexide diamine and
e) purifying the isohexide diamine from step d) to obtain a purified isohexide diamine, where step c) is performed before step d).

As has now surprisingly been found, the process according to the invention has provided a process which provides isohexide diamines, preferably ISODA, in sufficiently high purity, for example sufficient for subsequent use in polymer formation reactions and phosgenations for conversion into the corresponding diisocyanates.

### DESCRIPTION

According to the invention, the references to “comprising”, “containing”, etc. preferably denote “substantially consisting of” and very particularly preferably denote “consisting of”. The further embodiments mentioned in the claims and in the description may be combined arbitrarily, provided that the context does not clearly indicate that the opposite is the case.

“At least one”, as used herein, refers to 1 or more, for example 2, 3, 4, 5, 6, 7, 8, 9 or more. In connection with constituents of the compounds described herein, this FIGURE refers not to the absolute number of molecules, but rather to the nature of the constituent. “At least one isohexide diol” therefore means, for example, that only one type of compound or a plurality of different types of compounds of this type may be present, without specifying the amount of the individual compounds.

Numerical values specified herein without decimal places each refer to the full value specified with one decimal place. For example, “99%” signifies “99.0%”.

Numerical ranges given in the format “in/from x to y” include the values stated. If two or more preferred numerical ranges are given in this format, it is understood that all ranges arising from the combination of the various end points are likewise encompassed.

A “substantially bis-aminated isohexide derivative-containing product” is preferably understood in the present case to mean that the reaction product comprises the at least one bis-aminated isohexide derivative to an extent of >85% by weight, preferably to an extent of >90% by weight, particularly preferably to an extent of >95% by weight and very particularly preferably to an extent of >99% by weight and most preferably consists thereof.

In a first preferred embodiment, the at least one isohexide diol is isosorbitol.

In a further preferred embodiment, the at least one isohexide diamine is (3R,3aR,6S,6aR)-3,6-diamino-2,3,3a,5,6,6a-hexahydrofuro[3,2-b]furan (also called isosorbide diamine or ISODA).

### Step a)

An isohexide diol, preferably isosorbitol, is converted into a suitably substituted derivative for process step b) by methods known to those skilled in the art (cf. Organikum, Wiley-VCH, 22^{nd} Edition, 2000, p. 655ff) for example with sulfonic acids, sulfonic anhydrides or sulfonyl halides. Preference is given to sulfonic anhydrides and sulfonyl halides, particular preference is given to sulfonyl chlorides, and very particular preference is given to tosyl chloride.

In a further preferred embodiment, the reaction in step a) is effected with at least one sulfonic acid, one sulfonic anhydride and/or one sulfonyl halide, preferably with at least one sulfonic anhydride and/or one sulfonyl halide, particularly preferably with at least one sulfonyl chloride and very particularly preferably with at least tosyl chloride.

The reaction may be effected with or without use of solvents and auxiliaries; preference is given to working in a solvent, particularly preferably in pyridine. It is possible here to work in the temperature range between −50° C. and the boiling point of the solvent in question; preference is given to working at low temperature, between −20° C. and +20° C. The reaction time may also be varied over a wide range and is generally 12 to 120 h, preferably 72 to 120 h.

### Step b)

The at least one suitably substituted derivative from step a) may be aminated with all known aminating agents which later allow the bis-aminated isohexide formed in this process step to be converted into a primary amino group. Examples are ammonia and primary amines.

In the context of the present invention, phthalimides, such as potassium phthalimide, are excluded from the aminating agents to be used in step b).

In a further preferred embodiment, the amination in step b) is performed with at least one primary amine, preferably with at least one amine having a primary, benzylically bonded NH₂ group and preferably with at least benzylamine.

Primary amines that boil above 100° C., preferably above 150° C., at the reaction pressure are preferred. Particularly preferred are amines having primary, benzylically bonded NH₂ groups such as benzylamine, 2-methylbenzylamine, 3-methylbenzylamine, 4-methylbenzylamine, 2,3-dimethylbenzylamine, 2,4-dimethylbenzylamine, 2,5-dimethylbenzylamine, 2,3,4-trimethylbenzylamine, 1-naphthylmethylamine, 2-naphthylmethylamine, since the cleavage thereof in step d) forms alkyl aromatics that are easy to separate off; use is made of benzylamine in the particularly preferred case, the cleavage of which in step d) then forms toluene as alkyl aromatic to be separated off.

### Step c)

The reaction product mixture from step c) is separated preferably by extraction or by distillation or by combinations thereof. In the case of distillative separation, preference is given to the thin-film distillation method known to those skilled in the art, particularly the short-path distillation method.

### Step d)

The deprotection may in principle be effected according to generally known conditions.

In a further preferred embodiment, the deprotection in step d) is performed catalytically, preferably in the presence of at least one noble metal catalyst, particularly preferably by means of at least one metal from the eighth, ninth and tenth group of the Periodic Table and very particularly preferably with at least one palladium compound. Very particularly preferred is the combination of palladium hydroxide (for example 20% on activated carbon) and palladium (for example 10% on activated carbon).

The preferably used method of catalytic (reductive) debenzylation is known (ChemSusChem 2011, 4, 1823 1829) for the case of the preferred use in step b) of an amine having a primary, benzylically bonded NH₂ group. A number of noble metal catalysts can be used for this purpose. Metals from the eighth, ninth and tenth group of the Periodic Table are preferably used for this purpose; palladium compounds are particularly preferred and the combination of palladium hydroxide (for example 20% on activated carbon) and palladium (for example 10% on activated carbon) is very particularly preferred.

### Step e)

The reaction mixture from step d) is preferably separated, after removal of the optionally present catalyst and catalyst support material for example by means of solid/liquid separation methods such as preferably filtration or centrifugation, also by extraction or distillation or combinations thereof. In the case of distillative separation, preference is given to the thin-film distillation method, particularly the short-path distillation method, where the distillates obtained are particularly preferably subsequently fed to a fine distillation through a suitable column.

The process according to the invention has made it possible for the first time to access suitable compositions of isohexide diamines.

Therefore, the present invention further provides a composition comprising at least one isohexide diamine and <1000 ppm by weight, preferably <500 ppm by weight and particularly preferably <100 ppm by weight, of tricyclic 2,5-(amino)-2,5-dideoxy-1,4:3,6-dianhydromannitol and/or <1000 ppm by weight, preferably <500 ppm by weight and particularly preferably <100 ppm by weight, of N-benzylpyrrole. The isohexide diamine is preferably (3R,3aR,6S,6aR)-3,6-diamino-2,3,3a,5,6,6a-hexahydrofuro[3,2-b]furan (isosorbide diamine, ISODA).

The content of the at least one isohexide diamine, preferably of the ISODA, is determined by means of GC (FID) by the 100% method.

The content of tricyclic 2,5-(amino)-2,5-dideoxy-1,4:3,6-dianhydromannitol is determined by means of GC after calibration by the internal standard method (“IST method”).

The content of N-benzylpyrrole is also determined by means of GC after calibration by the internal standard method (“IST method”).

In addition and alternatively to the abovementioned composition according to the invention, the invention further provides a composition comprising a mixture of >90% to <100% by weight of an isohexide diamine, preferably (3R,3aR,6S,6aR)-3,6-diamino-2,3,3a,5,6,6a-hexahydrofuro[3,2-b]furan (isosorbide diamine, ISODA), and >0% to <10% by weight, preferably >0.5% to <8% by weight, of another isohexide diamine isomer, preferably (3S,3aR,6S,6aR)-3,6-diamino-2,3,3a,5,6,6a-hexahydrofuro[3,2-b]furan (also called “isoidide diamine” or “IDDA” in the present text), based on the two aforementioned isomeric diamines.

Preferably, the composition according to the invention comprises a mixture of >90% to <99.5% by weight, preferably >92% to <99.5% by weight, particularly preferably >92% to <99% by weight, of an isohexide diamine, preferably (3R,3aR,65,6aR)-3,6-diamino-2,3,3a,5,6,6a-hexahydrofuro[3,2-b]furan (isosorbide diamine, ISODA), and >0.5% to <10% by weight, preferably >0.5% to <8% by weight, particularly preferably >1% to <8% by weight, of another isohexide diamine isomer, preferably (3S,3aR,6S,6aR)-3,6-diamino-2,3,3a,5,6,6a-hexahydrofuro[3,2-b]furan (also called “isoidide diamine” or “IDDA” in the present text), based on the two aforementioned isomeric diamines.

Particularly preferably, the composition according to the invention comprises a mixture of >90% to <100% by weight, preferably >90% to <99.5% by weight, particularly preferably >92% to <99.5% by weight, very particularly preferably >92% to <99% by weight, of (3R,3aR,6S,6aR)-3,6-diamino-2,3,3a,5,6,6a-hexahydrofuro[3,2-b]furan (also referred to as “isosorbide diamine” or “ISODA”), and >0% to <10% by weight, preferably >0.5% to <10% by weight, particularly preferably >0.5% to <8% by weight, very particularly preferably >1% to <8% by weight, of (3S,3aR,65,6aR)-3,6-diamino-2,3,3a,5,6,6a-hexahydrofuro[3,2-b]furan (also called “isoidide diamine” or “IDDA” in the present text), based on the total amount of ISODA and IDDA.

In addition, the invention further provides a composition comprising a mixture of >90% to <99.5% by weight of an isohexide diamine, preferably (3R,3aR,6S,6aR)-3,6-diamino-2,3,3a,5,6,6a-hexahydrofuro[3,2-b]furan, and >0.5% to <10% by weight of another isohexide diamine isomer, preferably (3S,3aR,6S,6aR)-3,6-diamino-2,3,3a,5,6,6a-hexahydrofuro[3,2-b]furan, based on the two aforementioned isomeric isohexide diamines.

Preferably, the composition according to the invention comprises a mixture of >92% to <99.5% by weight, preferably >92% to <99% by weight, of an isohexide diamine, preferably (3R,3aR,65,6aR)-3,6-diamino-2,3,3a,5,6,6a-hexahydrofuro[3,2-b]furan (isosorbide diamine, ISODA), and >0.5% to <8% by weight, preferably >1% to <8% by weight, of another isohexide diamine isomer, preferably (3S,3aR,6S,6aR)-3,6-diamino-2,3,3a,5,6,6a-hexahydrofuro[3,2-b]furan (also called “isoidide diamine” or “IDDA” in the present text), based on the two aforementioned isomeric diamines.

Particularly preferably, the composition according to the invention comprises a mixture of >90% to <99.5% by weight, preferably >92% to <99.5% by weight, particularly preferably >92% to <99% by weight, of (3R,3aR,6S,6aR)-3,6-diamino-2,3,3a,5,6,6a-hexahydrofuro[3,2-b]furan (also referred to as “isosorbide diamine” or “ISODA”), and >0.5% to <10% by weight, preferably >0.5% to <8% by weight, particularly preferably >1% to <8% by weight, of (3S,3aR,6S,6aR)-3,6-diamino-2,3,3a,5,6,6a-hexahydrofuro[3,2-b]furan (also called “isoidide diamine” or “IDDA” in the present text), based on the total amount of ISODA and IDDA.

The proportions of the different isohexide diamine isomers, preferably ISODA and IDDA, are each determined by means of GC after calibration by the internal standard method (“IST method”).

The compositions according to the invention essentially comprise the abovementioned compounds. In this context, “essentially” means that the abovementioned compounds amount to preferably 95% by weight or more, particularly preferably 97% by weight or more and very particularly preferably 99% by weight or more, in each case based on the total weight of the compositions according to the invention. These contents are determined by gas chromatography (FID) by the 100% method, with the secondary components being determined by means of GC after calibration by the internal standard method (“IST method”). The compositions according to the invention may comprise further compounds, but they are preferably present in the smallest possible amount. However, the effort required for complete removal should be weighed against the influence on the end product.

Very particularly preferably, the composition according to the invention consists of >90% to <100% by weight, preferably >90% to <99.5% by weight, particularly preferably >92% to <99.5% by weight, very particularly preferably >92% to <99% by weight, of (3R,3aR,6S,6aR)-3,6-diamino-2,3,3a,5,6,6a-hexahydrofuro[3,2-b]furan (also referred to as “isosorbide diamine” or “ISODA”), and >0% to <10% by weight, preferably >0.5% by weight to <10% by weight, particularly preferably >0.5% by weight to <8% by weight, very particularly preferably >1% by weight to <8% by weight, of (3S,3aR,6S,6aR)-3,6-diamino-2,3,3a,5,6,6a-hexahydrofuro[3,2-b]furan (also called “isoidide diamine” or “IDDA” in the present text).

The compositions according to the invention are liquid at 23° C. and prove to be crystallization-stable even when stored at 0° C. for a relatively long time. This is particularly surprising since only solid isohexide diamines are known in the prior art, see for example ChemSusChem 2011, 4, 1823-1829, where the compounds are described as crystalline solids.

This results in the further advantage that the compositions according to the invention are able to be pumped and can therefore be used particularly well for industrial-scale processes for example in the chemical industry.

The present invention therefore further provides for the use of a composition according to the invention for preparing polymer precursors and/or polymers, preferably monomeric diisocyanates, polyisocyanates and/or polyurethanes.

The present invention likewise provides a polymer precursor or polymer comprising the reaction product of a composition according to the invention with at least one compound that is reactive toward amino groups. The polymer precursors are preferably monomeric diisocyanates and/or polyisocyanates. The polymer is preferably a polyurethane.

If, for example, the at least one compound that is reactive toward amino groups is phosgene, then the corresponding monomeric diisocyanate is obtained. If the isohexide diamine is, preferably the compositions according to the invention are, reacted either with carboxylic acids or carbonyl halides with amide formation, polyamide is obtained as polymer in the context of the invention.

Alternatively, also included is a reaction with commercially available epoxy curing agents to form epoxy resins as polymer in the context of the invention. A further option, which is preferred herein, is the reaction of the isohexide diamines, preferably of the compositions according to the invention, to form monomeric diisocyanates and/or polyisocyanates which are then reacted with compounds that are reactive toward isocyanate groups, preferably with polyols, to form polyurethanes. Furthermore, it is possible to react the isohexide diamines, preferably the compositions according to the invention, with monomeric diisocyanates and/or polyisocyanates based on these or any other amines, which then results in polyureas. Combinations of the aforementioned reactions are also possible according to the invention.

The invention is elucidated in more detail hereinafter using examples, but without being limited thereto.

### EXAMPLES

Unless otherwise noted, all amounts, such as “parts”, and all percentages are based on weight.

NMR spectroscopy measurements were conducted on the Bruker DPX 400 or DRX 700 instruments on approx. 5% (1H NMR) or approx. 50% (13C NMR) samples in dry CDCl₃, unless noted otherwise, at 400 or 700 MHZ (1H NMR) or 100 or 176 MHz (13C NMR). The reference employed for the ppm scale was tetramethylsilane in the solvent with 1H NMR chemical shift 0 ppm. Alternatively, CHCI₃ present in the NMR solvent was used as reference signal (7.26 ppm, 1H NMR), or the solvent signal itself (average signal of the 1:1:1 triplet at 77.0 ppm in the 13C NMR).

GC-MS was performed using the Agilent GC6890, equipped with an MN 725825.30 Optima-5 MS Accent capillary column (30 m, 0.25 mm internal diameter, 0.5 μm film layer thickness) and a 5973 mass spectrometer as detector with helium as transport gas (constant flow rate of 2 ml/min). The column temperature was initially 60° C. (2 min) and was then increased gradually by 8K/min to 360° C. The GC-MS detection used electron impact ionization with 70 eV ionization energy. The injector temperature chosen was 250° C.

The FIGURES for the proportions by weight of the isohexide diamines were determined by gas chromatography (FID) by the 100% method, with the secondary components being determined by means of GC after calibration by the internal standard method (“IST method”). The reference substance employed for the IST method was tetradecane.

The isosorbitol used is a product from Roquette, 62136 Lestrem, France. All other commercially available chemicals were obtained from Merck.

### Example 1

### Preparation of 1,4:3,6-Dianhydro-2,5-Di-O-p-Tosyl-d-Sorbitol

200 parts of isosorbitol and 600 parts of pyridine were initially charged under nitrogen and cooled to 0° C. with stirring. Subsequently, a solution of 522 parts of p-toluenesulfonyl chloride in 1400 parts of pyridine was added dropwise with further external cooling and, after complete addition, stirring was continued for a further thirty minutes at an internal temperature of 0° C. The reaction mixture was left to stand for 78 hours at room temperature, then was poured into 10 000 parts of water-ice mixture and this mixture was then left to stand for a further eight hours.

Subsequently, the solid which settled out was filtered off, washed with 3×400 parts of water, 0.2M HCl and again with 3×400 parts of water and then recrystallized from ethanol. The ratio of product (moist) to ethanol here was approx. 1 to 2. The bis-tosylate dissolves completely at boiling and precipitates on cooling without any problems. The product, dried under reduced pressure at 50° C., is obtained as a while solid with an 86% yield.

### Example 2

### Preparation of 2,5-Bis(Benzylamino)-2,5-Dideoxy-1,4:3,6-Dianhydro-d-Sorbitol

660 parts of 1,4:3,6-dianhydro-2,5-di-O-p-tosyl-d-sorbitol, obtained in accordance with Example 1, and 933.5 parts of benzylamine were initially charged under nitrogen and stirred for four hours at a bath temperature of 185° C. The result was a dark red, clear solution. On cooling to approx. 70-80° C., the benzylammonium tosylate formed precipitated out in crystalline form. At this temperature, the reaction mixture was admixed, with stirring, with 1250 parts of ethyl acetate in order to improve the stirrability. Subsequently, the precipitate was filtered off and the filter residue was washed with 1250 parts of ethyl acetate and discarded.

The ethyl acetate was removed from the combined organic phases. 1050 parts of a dark red, viscous liquid remained.

### Example 3 (Non-Inventive)

### Direct Further Processing of the Product Mixture Obtained in Accordance with Example 2

The liquid mixture obtained in Example 2 was virtually quantitatively freed of excess benzylamine by means of vacuum distillation and the remaining distillation residue (645 parts), analogously to the procedure described in Example 5, was subject to catalytic debenzylation without further purification.

The crude product from the catalytic debenzylation was then rectified on a 1 m-long randomly packed column filled with Interpack random packings #1 and fitted reflux divider at a reflux ratio of 1:10. None of the distillate fractions obtained contained less than 1000 ppm by weight of the tricyclic 2,5-(amino)-2,5-dideoxy-1,4:3,6-dianhydromannitol, which boils at a somewhat lower temperature than the main component, ISODA, and is present in solid form at room temperature and therefore constantly clogged the reflux condenser, the distillate outlet conduits and the vacuum conduits.

The liquid N-benzylpyrrole also formed has a slightly higher boiling point than the 2,5-(amino)-2,5-dideoxy-1,4:3,6-dianhydromannitol and was therefore even more difficult to separate from the main component. It was also not possible in this way to deplete N-benzylpyrrole to proportions below 1000 ppm by weight in the final ISODA. The FIGURES for the proportions by weight of the secondary components were determined by means of GC after calibration with authentic material by the internal standard method (the reference substance employed was tetradecane).

### Example 4 (Inventive)

### Further Processing of the Product Mixture Obtained in Accordance with Example 2 Following Prior Distillation

A product mixture (1050 parts) obtained in accordance with Example 2 was separated into two fractions-distillate and distillation residue—over the course of 6 hours at 180° C. and 0.1 mbar by means of a thin-film evaporator of the short-path evaporator type. The distillate (550 parts) consisted predominantly of excess benzylamine, 2,5-(benzylamino)-2,5-dideoxy-1,4:3,6-dianhydro-d-mannitol and N-benzylpyrrole and was then separately worked up by distillation to give pure benzylamine, which was reused.

The brown distillation residue (500 parts of crude 2,5-bis(benzylamino)-2,5-dideoxy-1,4:3,6-dianhydro-d-sorbitol), which was highly viscous at room temperature, was combined with several analogously processed batches and processed further as described below.

### Example 5 (Inventive)

### Further Processing of the Product Mixture Obtained in Accordance with Example 4 Following Prior Distillation

2476 parts of crude 2,5-bis(benzylamino)-2,5-dideoxy-1,4:3,6-dianhydro-d-sorbitol (combined thin-film distillation residues) were dissolved in 1000 parts of methanol and, together with 100 parts of palladium hydroxide (20% on activated carbon) and 100 parts of palladium (10% on activated carbon), were placed in a hydrogenation reactor under nitrogen. Inertization was then performed (3×injection of nitrogen at 5 bar and discharge), a pressure test (80 bar of nitrogen) was carried out with the stirrer switched off, and the reactor was charged with hydrogen (20 bar).

With stirring, the temperature was slowly increased to 60° C. and the hydrogen pressure was increased to 40 bar. Hydrogenation was performed under these conditions until hydrogen uptake was quantitative (approx. 24 h). The reactor was then cooled, decompressed and opened. The solution was very substantially freed of the catalyst and the activated carbon under pressure using a fine filter. The filtrate was very substantially freed of the solvent under reduced pressure and the remaining residue (approx. 1100 parts) was then rectified on a 1 m-long randomly packed column filled with Interpack random packings #1 and fitted reflux divider at a reflux ratio of 1:10.

Without any deposition of solids in the distillate paths of the plant, after removing a few grams of a liquid forerun which, according to the GC, was free of N-benzylpyrrole, tricyclic 2,5-(amino)-2,5-dideoxy-1,4:3,6-dianhydromannitol and also free of benzylamine and already contained >90% of (3R,3aR,6S,6aR)-3,6-diamino-2,3,3a,5,6,6a-hexahydrofuro[3,2-b]furan (ISODA), 500 parts (45% of theory) of around 96% pure ISODA were obtained, at a top temperature of 100° C. and pressure of 0.3 mbar; the remainder to 100% was essentially IDDA.

1. A process for preparing isohexide diamines from isohexide diols, comprising the following steps:
a) reacting at least one isohexide diol to obtain at least one suitably substituted derivative,
b) aminating the at least one suitably substituted derivative from step a) to obtain a reaction product mixture,
c) separating the reaction product mixture from step b) to obtain a substantially bis-aminated isohexide-containing product,
d) deprotecting the substantially bis-aminated isohexide-containing product purified in step c) to obtain an isohexide diamine and
e) purifying the isohexide diamine from step d) to obtain a purified isohexide diamine, where step c) is performed before step d).

2. The process as claimed in claim 1, wherein the at least one isohexide diol is (3R,3aR,6S,6aR)-3,6-dihydroxy-2,3,3a,5,6,6a-hexahydrofuro[3,2-b]furan.

3. The process as claimed in claim 1, wherein the at least one isohexide diamine is (3R,3aR,6S,6aR)-3,6-diamino-2,3,3a,5,6,6a-hexahydrofuro[3,2-b]furan.

4. The process as claimed in claim 1, wherein the reaction in step a) is effected with at least one sulfonic acid, one sulfonic anhydride and/or one sulfonyl halide.

5. The process as claimed in claim 1, wherein the amination in step b) is performed with at least one primary amine.

6. The process as claimed in claim 1, wherein the separation in step c) is effected by extraction, by distillation or by combinations thereof.

7. The process as claimed in claim 1, wherein the deprotection in step d) is performed catalytically.

8. The process as claimed in claim 1, wherein the purification in step e), after removal of the optionally present catalyst and catalyst support material by means of solid/liquid separation methods is effected by extraction or distillation or combinations thereof.

9. A composition comprising a mixture of >90% to <99.5% by weight of an isohexide diamine and >0.5% to <10% by weight of another isohexide diamine isomer based on the two aforementioned isomeric isohexide diamines.

10. A method for preparing polymer precursors and/or polymers comprising providing the composition as claimed in claim 9.

11. A polymer precursor or polymer comprising the reaction product of a composition as claimed in claim 9 with at least one compound that is reactive toward amino groups.

12. The polymer precursor or polymer as claimed in claim 11, wherein the polymer precursor or polymer is a monomeric diisocyanate and/or polyisocyanate and/or polyurethane.

13. The process as claimed in claim 4, wherein the reaction in step a) is effected with at least one sulfonic anhydride and/or one sulfonyl halide.

14. The process as claimed in claim 5, wherein the amination in step b) is performed with at least one amine having a primary, benzylically bonded NH₂ group.

15. The process as claimed in claim 6, wherein the separation in step c) is effected by distillation.

16. The process as claimed in claim 7, wherein the deprotection in step d) is performed by means of at least one noble metal catalyst.

17. The composition as claimed in claim 9, wherein the isohexide diamine is (3R,3aR,6S,6aR)-3,6-diamino-2,3,3a,5,6,6a-hexahydrofuro[3,2-b]furan and the another isohexide diamine isomer is (3S,3aR,6S,6aR)-3,6-diamino-2,3,3a,5,6,6a-hexahydrofuro[3,2-b]furan.
